(19) United States
(12) Patent Application Publication
(10) Pub. No.: US 2024/0293113 A1
(43) Pub. Date: Sep. 5, 2024

(54) RETRACTOR DEVICE

(71) Applicant: Foot Innovations, LLC, Tampa, FL (US)

(72) Inventors: Roy W. Sanders, Tampa, FL (US); Sergio Gutierrez, Tampa, FL (US)

(73) Assignee: Foot Innovations, LLC, Tampa, FL (US)

(21) Appl. No.: 18/594,351

(22) Filed: Mar. 4, 2024

Related U.S. Application Data
(60) Provisional application No. 63/488,464, filed on Mar. 3, 2023.

(51) Int. Cl. A61B 17/02; A61B 17/00; A61B 17/56; A61B 90/00; A61F 2/46

(52) U.S. Cl. CPC A61B 17/025; A61F 2/4606; A61B 2017/0042; A61B 2017/564; A61B 2090/3966

(57) ABSTRACT

A surgical retractor device includes a body and a handle. The body having one or more curved profiles with an outer convex surface and including a peripheral edge including a top edge section including a first top edge portion and a second top edge portion; a first side edge portion; a first transition edge portion between the first top edge portion and the first side edge portion, the first transition edge portion defining a first radius; a bottom edge section; a second side edge portion; and a second transition edge portion between the second top edge portion and the second side edge portion, the second transition edge defining a second radius larger than the first radius. The handle has a proximal end and a distal end, the distal end extending from at least one of the top edge section, the first transition edge portion, or the first side edge portion.

### RELATED APPLICATIONS

This application claims priority to co-pending U.S. Provisional application having Ser. No. 63/488,464 filed on Mar. 3, 2023, the contents of which are hereby incorporated by reference in their entirety.

### TECHNICAL FIELD

The present disclosure relates generally to retractor devices. More particularly, the disclosure relates retractor devices used in surgery (e.g., ankle surgery).

### BACKGROUND

During various surgeries, cutting jigs may be used to remove structure from a desired location in the patient's body. For example, during ankle surgery (e.g., total ankle replacement, total ankle arthroplasty, ankle fusion, etc.) a cutting jig may be used to remove bone structure in the respective ankle (e.g., a portion of the tibia, a portion of the talus, etc.).

### SUMMARY

One embodiment relates to a surgical retractor device. The surgical retractor device including a body having one or more curved profiles with an outer convex surface and including a peripheral edge, wherein the peripheral edge of the body includes: a top edge section of the peripheral edge including a first top edge portion and a second top edge portion; a first side edge portion substantially perpendicular to the first top edge portion; a first transition edge portion between the first top edge portion and the first side edge portion, the first transition edge portion defining a first radius; a bottom edge section; an angled edge portion extending at an angle between the first side edge portion and the bottom edge section; a second side edge portion opposite the first side edge portion and substantially perpendicular to the second top edge portion; and a second transition edge portion between the second top edge portion and the second side edge portion, the second transition edge defining a second radius, wherein the second radius of the second transition edge portion is larger than the first radius of the first transition edge portion; and a handle having a proximal end and a distal end, the distal end of the handle extending from at least one of the top edge section, the first transition edge portion, or the first side edge portion.

Another embodiment relates to a surgical retractor device. The surgical retractor device includes a body defining: a first profile within a first plane; a second profile within a second plane perpendicular to the first plane; a top edge section; a side edge portion substantially perpendicular to the top edge section; and a transition edge portion between the top edge section and the side edge portion, wherein the first profile includes a curved portion, a flat portion, and a transition portion, wherein the curved portion of the first profile defines a convex outer surface of the body, wherein the transition portion of the first profile defines a concave outer surface of the body, and wherein the second profile defines a continuously convex outer surface of the body; and a handle extending from at least one of the top edge section, the transition edge portion, or the side edge portion.

Still another embodiment relates to a method of performing a total ankle replacement surgery. The method includes making a first incision on a medial side of an ankle area of a patient; inserting a retractor device into the patient via the first incision, the retractor device including a handle and a body having an outer convex surface; adjusting a position of the retractor device towards a posterior side of a talus of the patient; and inserting via a second incision on an anterior side of the ankle area of the patient a surgical instrument to perform a surgical procedure involving at least one of a calcaneus or the talus of the patient.

These and other features of various embodiments can be understood from a review of the following detailed description in conjunction with the accompanying drawings.

It is to be understood that both the foregoing general description and the following detailed description are exemplary and explanatory and are not restrictive of the present invention, as claimed.

### BRIEF DESCRIPTION OF THE DRAWINGS

FIG. 1 is a perspective view of a retractor and an ankle joint, according to an example embodiment.

FIG. 2 is another perspective view of the retractor and the ankle joint of FIG. 1.

FIG. 3 is another perspective view of the retractor and the ankle joint of FIG. 1.

FIG. 4 is another perspective view of the retractor and the ankle joint of FIG. 1.

FIG. 5 is a front view of the retractor of FIG. 1.

FIG. 6 is a rear view of the retractor of FIG. 1.

FIG. 7 is a side view of the retractor of FIG. 1.

FIG. 8 is another side view of the retractor of FIG. 1.

FIG. 9 is a top view of the retractor of FIG. 1.

FIG. 10 is a bottom view of the retractor of FIG. 1.

FIG. 11 is a perspective view of another retractor and an ankle joint, according to an example embodiment.

FIG. 12 is another perspective view of the retractor and the ankle joint of FIG. 11.

FIG. 13 is another perspective view of the retractor and the ankle joint of FIG. 11.

FIG. 14 is another perspective view of the retractor and the ankle joint of FIG. 11.

FIG. 15 is a front view of the retractor of FIG. 11.

FIG. 16 is a rear view of the retractor of FIG. 11.

FIG. 17 is a side view of the retractor of FIG. 11.

FIG. 18 is another side view of the retractor of FIG. 11.

FIG. 19 is a bottom view of the retractor of FIG. 11.

FIG. 20 is a top view of the retractor of FIG. 11.

FIG. 21 is a front view of another retractor, according to an example embodiment.

FIG. 22 is a rear view of the retractor of FIG. 21.

FIG. 23 is a side view of the retractor of FIG. 21.

FIG. 24 is another side view of the retractor of FIG. 21.

FIG. 25 is a bottom view of the retractor of FIG. 21.

FIG. 26 is a top view of the retractor of FIG. 21.

FIG. 27 is a perspective view of another retractor, according to an example embodiment.

FIG. 28 is a front view of the retractor of FIG. 27.

FIG. 29 is a rear view of the retractor of FIG. 27.

FIG. 30 is a perspective view of another retractor, according to an example embodiment.

FIG. 31 is a front view of the retractor of FIG. 30.

FIG. 32 is a rear view of the retractor of FIG. 30.

FIG. 33 is a perspective view of another retractor, according to an example embodiment.

FIG. 34 is a front view of the retractor of FIG. 33.

FIG. 35 is a rear view of the retractor of FIG. 33.

FIG. 36 is a front view of a body of a retractor, according to an example embodiment.

FIG. 37 is a front view of another body of a retractor, according to an example embodiment.

FIG. 38 is a front view of another body of a retractor, according to an example embodiment.

FIG. 39 is a front view of another body of a retractor, according to an example embodiment.

FIG. 40 is a front view of another body of a retractor, according to an example embodiment.

FIG. 41 is a front view of another body of a retractor, according to an example embodiment.

FIG. 42 is a front view of another retractor, according to an example embodiment.

FIG. 43 is a front perspective view of another retractor, according to an example embodiment.

FIG. 44 is a rear perspective view of the retractor of FIG. 43, according to an example embodiment.

FIG. 45 is a front view of the retractor of FIG. 43, according to an example embodiment.

FIG. 46 is a rear view of the retractor of FIG. 43, according to an example embodiment.

FIG. 47 is a left side view of the retractor of FIG. 43, according to an example embodiment.

FIG. 48 is a right side view of the retractor of FIG. 43, according to an example embodiment.

FIG. 49 is a top view of the retractor of FIG. 43, according to an example embodiment.

FIG. 50 is a bottom side view of the retractor of FIG. 43, according to an example embodiment.

FIG. 51 is a section view of a body of the retractor of FIG. 43 within a first plane, according to an example embodiment.

FIG. 52 is a section view of the body of the retractor of FIG. 43 within a second plane, according to an example embodiment.

FIG. 53 is a front perspective view of another retractor, according to an example embodiment.

FIG. 54 is a rear perspective view of the retractor of FIG. 53, according to an example embodiment.

FIG. 55 is a front view of the retractor of FIG. 53, according to an example embodiment.

FIG. 56 is a rear view of the retractor of FIG. 53, according to an example embodiment.

FIG. 57 is a left side view of the retractor of FIG. 53, according to an example embodiment.

FIG. 58 is a right side view of the retractor of FIG. 53, according to an example embodiment.

FIG. 59 is a top view of the retractor of FIG. 53, according to an example embodiment.

FIG. 60 is a bottom side view of the retractor of FIG. 53, according to an example embodiment.

FIG. 61 is a section view of a body of the retractor of FIG. 53 within a first plane, according to an example embodiment.

FIG. 62 is a section view of the body of the retractor of FIG. 53 within a second plane, according to an example embodiment.

FIG. 63 is a flow diagram of a method of performing a total ankle replacement surgery, according to an example embodiment.

### DETAILED DESCRIPTION

The following detailed description and the appended drawings describe and illustrate various exemplary external fixation systems, methods, and components. The description and drawings are exemplary in nature and are provided to enable one skilled in the art to make and use one or more exemplary external fixation systems and/or components, and/or practice one or more exemplary methods. They are not intended to limit the scope of the claims in any manner.

While the systems, methods, and components described herein are exemplified by systems and methods for using a retractor during ankle surgery, the systems, methods, and components described and illustrated herein can be used to treat any suitable ailment or joint within the body of an animal, including, but not limited to, humans. Skilled artisans will be able to select a suitable ailment and/or joint within the body of an animal to utilize a system and/or method described herein according to a particular embodiment based on various considerations, including the type of ailment and/or the structural arrangement at a treatment site. Example joints considered suitable to utilize a system, method, and/or component described herein include, but are not limited to, the elbow joint, and the knee joint.

According to various example embodiments, cutting jigs are used to remove structure from a patient as a part of the surgery. For example, during ankle surgery (e.g., total ankle replacement, total ankle arthroplasty, ankle fusion, etc.) a cutting jig may be used to remove bone structure in the respective ankle (e.g., a portion of the tibia, a portion of the talus, etc.).

As described herein, a retractor may be inserted into the patient (e.g., proximate the ankle joint) to protect nearby tissues (e.g., muscle, tendons, arteries, etc.). As is described further herein, the retractor may prevent the cutting jig from cutting or removing the nearby tissue that is not intended to be cut by the cutting jig.

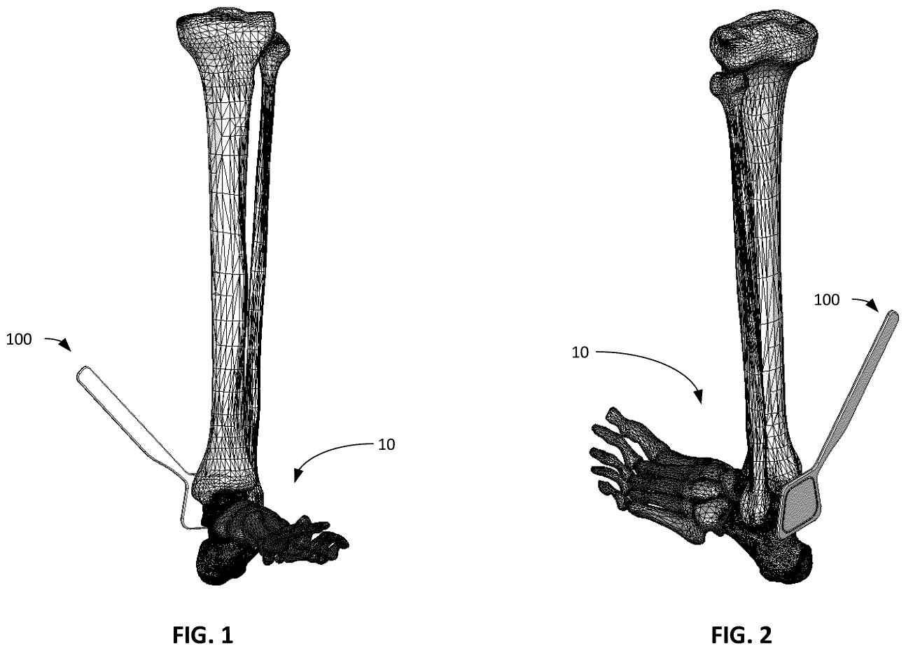

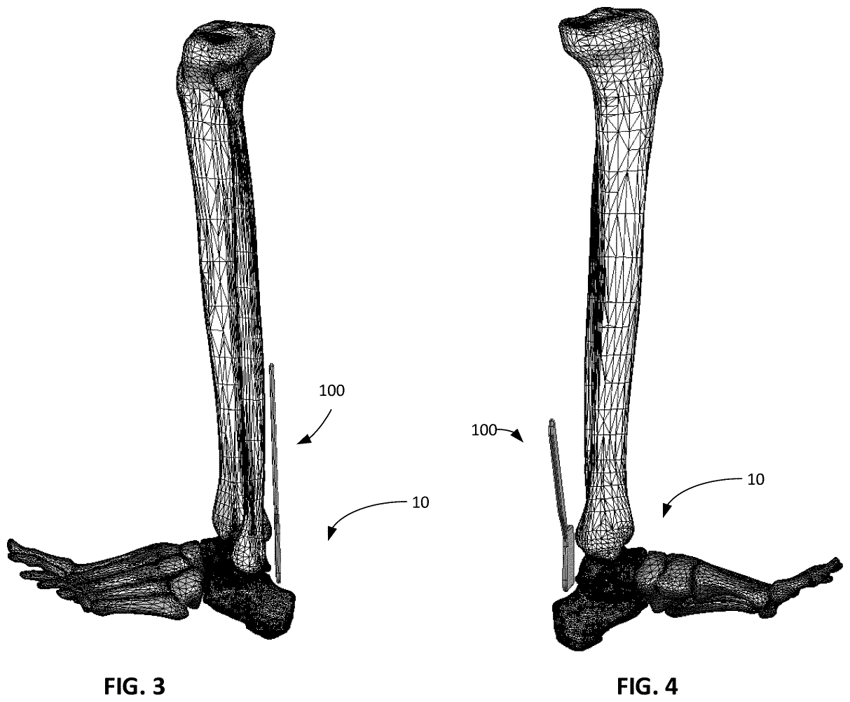

Referring now to FIGS. 1-4, perspective views of a retractor 100 and an ankle joint 10 are shown, according to an example embodiment. As shown, the retractor 100 is positioned proximate a posterior portion of the ankle joint 10. According to various examples, an incision may be made in the patient's leg area, foot area, a medial side of an ankle area to insert the retractor 100 into the position shown in FIGS. 1-4. According to various embodiments, the retractor prevents a cutting jig from damaging the Achilles tendon. For example, a cutting jig may be inserted into the patient proximate an anterior portion of the ankle joint 10 and structure (e.g., a portion of the tibia, a portion of the talus, etc.) may be removed from the patient. The retractor 100 may prevent the cutting jig from traveling beyond an undesired location in the ankle joint 10, thereby protecting tissue positioned posteriorly of the retractor 100.

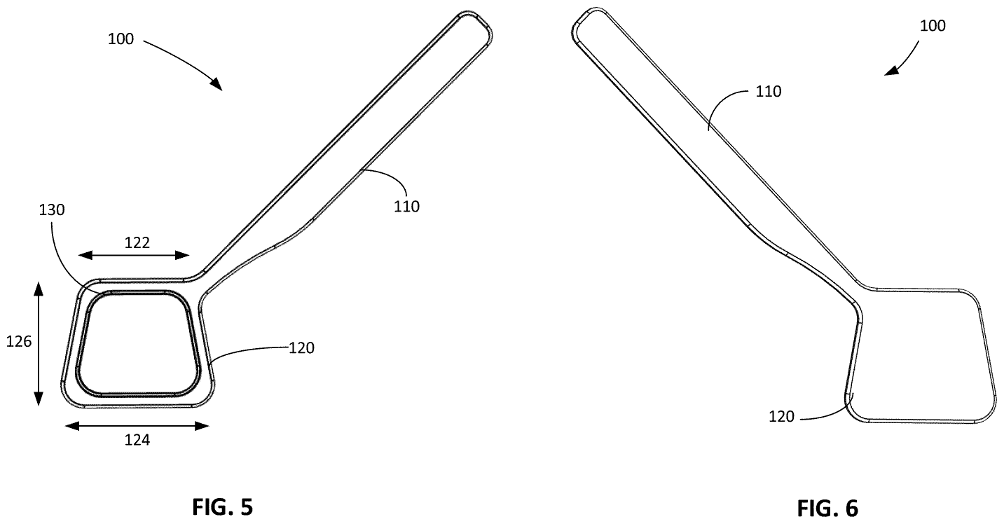

Referring now to FIGS. 5-10, a front view, rear view, side views, a top view, and a bottom view of the retractor 100 are shown, according to an example embodiment. As shown, the retractor 100 includes a handle 110 coupled to a body 120. According to various embodiments, the handle 110 is integrally formed with the body 120. According to other embodiments, the handle 110 is removably coupled to the body 120 (e.g., via a clip, threads, snap feature, etc.).

The handle 110 is shown coupled to an upper corner of the body 120, however, according to other embodiments, the location of the handle 110 may be located along other locations of the top and lateral edge of the body 120. According to various embodiments, the position of the handle 110 relative to the body 120 may allow the retractor to be inserted into the desired location (e.g., shown in FIGS. 1-4) of the patient without the need to cut any tendons.

As shown, the body 120 defines a height 126, a top width 122 and a bottom width 124. According to various embodiments, the retractor 100 may be included in a kit of retractors, each being sized differently such that retractor 100 selected is chosen based on the size of the patient's ankle joint 10. According to various embodiments, the height 126 is less than or equal to 50 mm.

The height 126 may be less than or equal to 45 mm. The height 126 may be less than or equal to 40 mm. The height 126 may be less than or equal to 35 mm. The height 126 may be less than or equal to 30 mm. The height 126 may be less than or equal to 25 mm. The height 126 may be less than or equal to 20 mm. The height 126 may be less than or equal to 15 mm. The height 126 may be less than or equal to 10 mm. The height 126 may be less than or equal to 5 mm.

The top width 122 may be greater than or equal to 5 mm. The top width 122 may be greater than or equal to 10 mm. The top width 122 may be greater than or equal to 15 mm. The top width 122 may be greater than or equal to 20 mm. The top width 122 may be greater than or equal to 25 mm. The top width 122 may be greater than or equal to 30 mm. The top width 122 may be greater than or equal to 35 mm. The top width 122 may be greater than or equal to 40 mm.

The top width 122 may be less than or equal to 45 mm. The top width 122 may be less than or equal to 40 mm. The top width 122 may be less than or equal to 35 mm. The top width 122 may be less than or equal to 30 mm. The top width 122 may be less than or equal to 25 mm. The top width 122 may be less than or equal to 20 mm. The top width 122 may be less than or equal to 15 mm. The top width 122 may be less than or equal to 10 mm. The top width 122 may be less than or equal to 5 mm.

The bottom width 124 may be less than or equal to 45 mm. The bottom width 124 may be less than or equal to 40 mm. The bottom width 124 may be less than or equal to 35 mm. The bottom width 124 may be less than or equal to 30 mm. The bottom width 124 may be less than or equal to 25 mm. The bottom width 124 may be less than or equal to 20 mm. The bottom width 124 may be less than or equal to 15 mm. The bottom width 124 may be less than or equal to 10 mm. The bottom width 124 may be less than or equal to 5 mm.

The bottom width 124 may be greater than or equal to 5 mm. The bottom width 124 may be greater than or equal to 10 mm. The bottom width 124 may be greater than or equal to 15 mm. The bottom width 124 may be greater than or equal to 20 mm. The bottom width 124 may be greater than or equal to 25 mm. The bottom width 124 may be greater than or equal to 30 mm. The bottom width 124 may be greater than or equal to 35 mm. The bottom width 124 may be greater than or equal to 40 mm.

As shown, the retractor 100 includes a groove 130. The groove 130 is configured to receive an indicator. The indicator may be radiolucent (e.g., if the body 120 is radiopaque) or radiopaque (e.g., if the body 120 is radiolucent) such that the location and orientation of the retractor 100 can be identified in an image (e.g., an x-ray).

According to other embodiments, the indicator is integrally formed in the body 120. For example, the body 120 may be formed of a plastic material that is over-molded onto an indicator (e.g., a radiolucent wire).

According to various embodiments, the retractor 100 includes metal material. Further, the retractor 100 may include radiolucent and/or radiopaque plastic material. The retractor 100 may be disposable. Alternatively, the retractor 100 may be formed of a material that can be sanitized and used again.

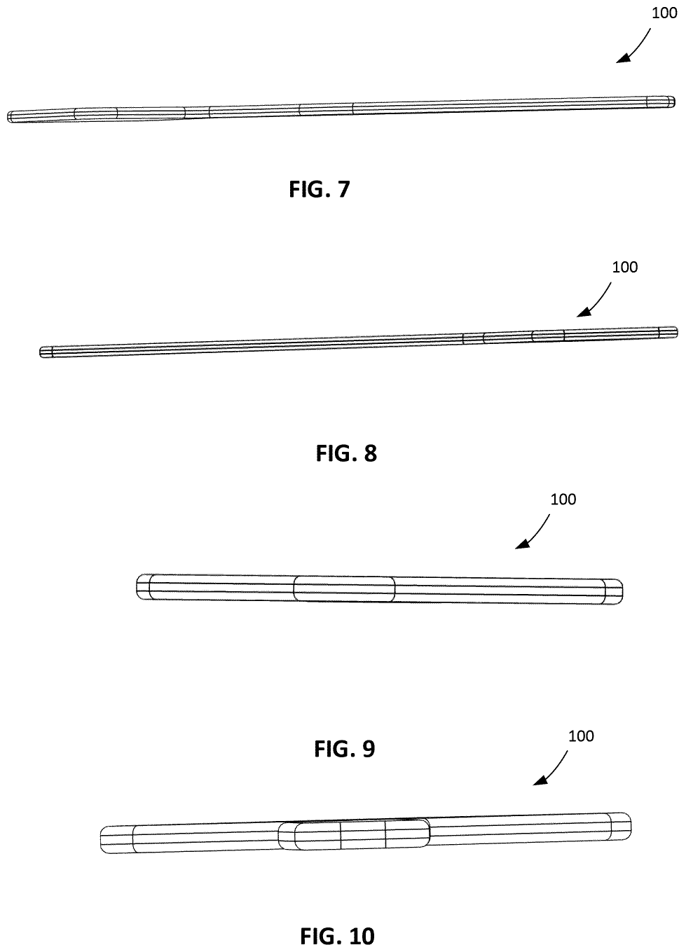

As best shown in FIGS. 7-10, the retractor 100 is substantially flat. According to various embodiments, such an arrangement allows the retractor to be used in conjunction with both the right ankle and the left ankle.

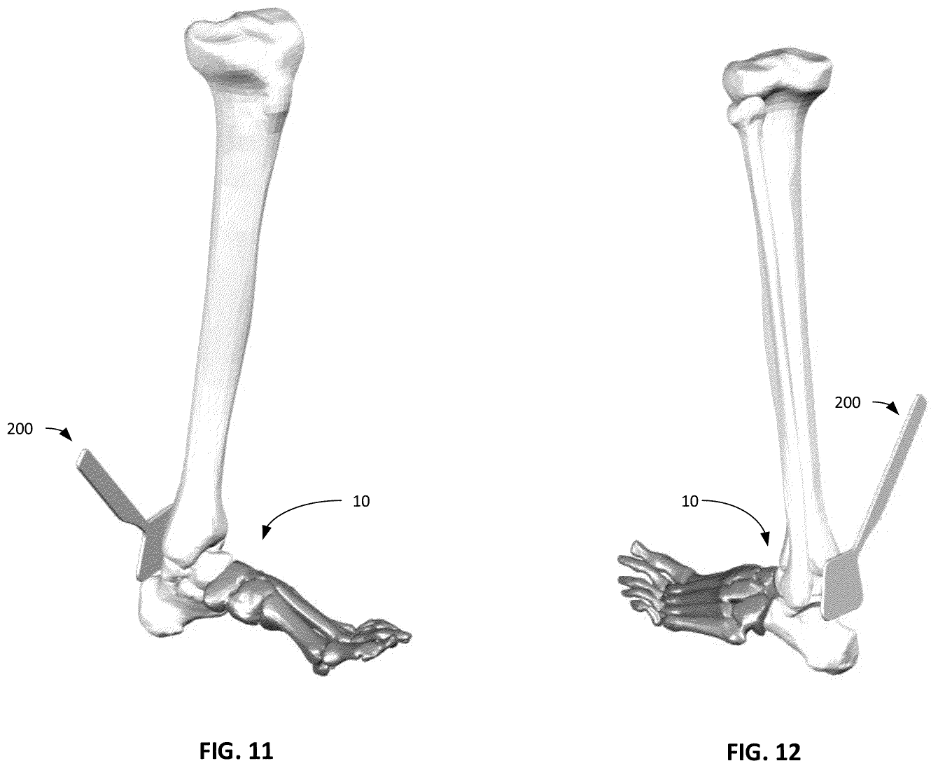

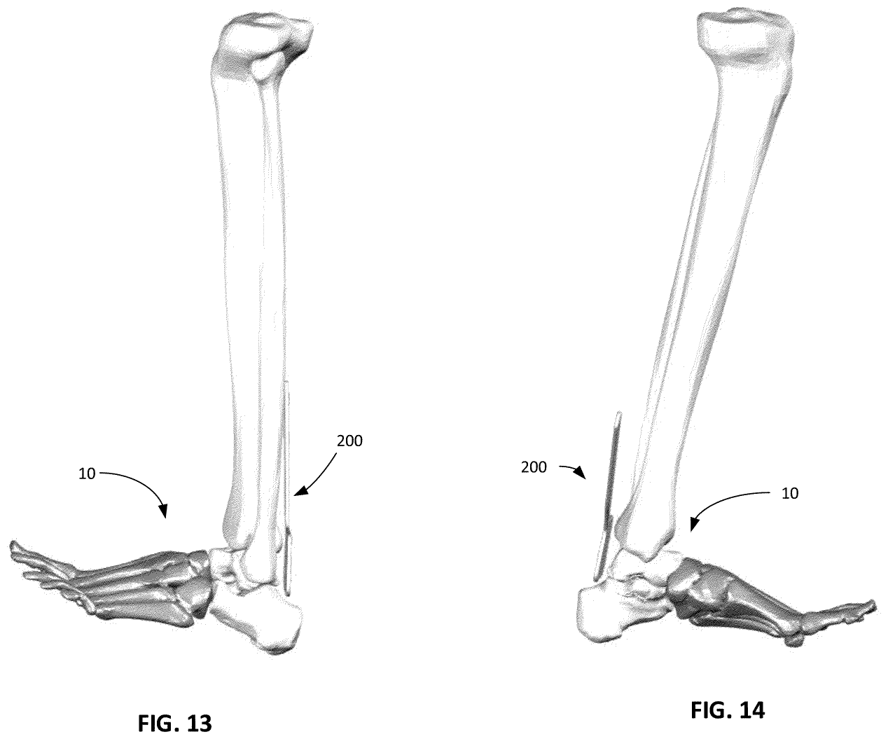

Referring now to FIGS. 11-14, perspective views of a retractor 200 and an ankle joint 20 are shown, according to an example embodiment. As shown, the retractor 200 is positioned proximate a posterior portion of the ankle joint 20. According to various examples, an incision may be made in the patient's leg/foot area to insert the retractor 200 into the position shown in FIGS. 11-14. According to various embodiments, the retractor prevents a cutting jig from damaging the Achilles tendon. For example, a cutting jig may be inserted into the patient proximate an anterior portion of the ankle joint 20 and structure (e.g., a portion of the tibia, a portion of the talus, etc.) may be removed from the patient. The retractor 200 may prevent the cutting jig from traveling beyond an undesired location in the ankle joint 20, thereby protecting tissue positioned posteriorly of the retractor 200.

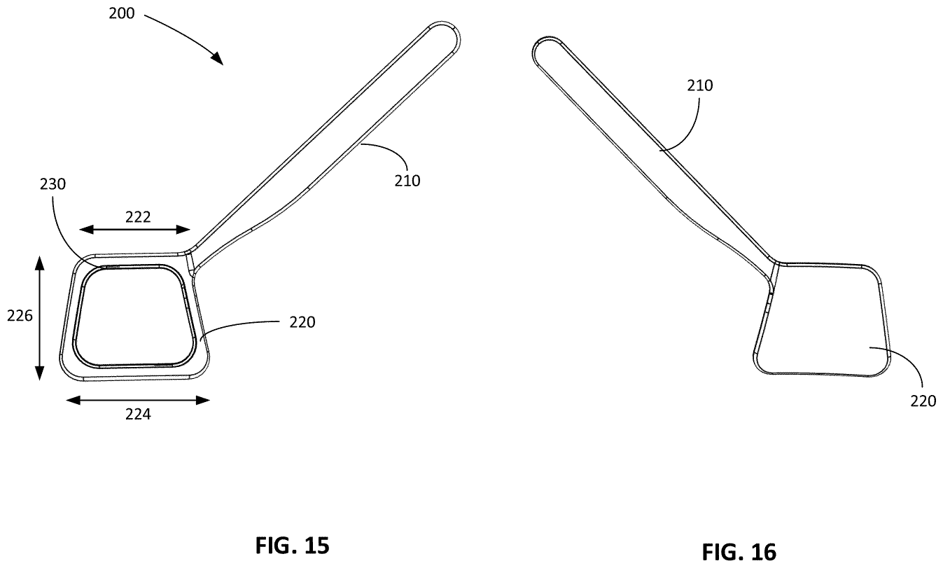

Referring now to FIGS. 15-20, a front view, rear view, side views, a top view, and a bottom view of the retractor 200 are shown, according to an example embodiment. As shown, the retractor 200 includes a handle 210 coupled to a body 220. According to various embodiments, the handle 210 is integrally formed with the body 220. According to other embodiments, the handle 210 is removably coupled to the body 220 (e.g., via a clip, threads, snap feature, etc.).

The handle 210 is shown coupled to an upper corner of the body 220, however, according to other embodiments, the location of the handle 210 may be located along other locations of the top and lateral edge of the body 220. According to various embodiments, the position of the handle 210 relative to the body 220 may allow the retractor to be inserted into the desired location (e.g., shown in FIGS. 11-14) of the patient without the need to cut any tendons.

As shown, the body 220 defines a height 226, a top width 222 and a bottom width 224. The height 226, the top width 222, and the bottom width 224 may be adapted for use with any number of cutting jigs. According to various embodiments, the retractor 200 may be included in a kit of retractors, reaching being sized differently such that retractor 200 selected is chosen based on the size of the patient's ankle joint. According to various embodiments, the height 226 is less than or equal to 50 mm.

The 226 height may be less than or equal to 45 mm. The 226 height may be less than or equal to 40 mm. The 226 height may be less than or equal to 35 mm. The 226 height may be less than or equal to 30 mm. The 226 height may be less than or equal to 25 mm. The 226 height may be less than or equal to 20 mm. The 226 height may be less than or equal to 15 mm. The 226 height may be less than or equal to 10 mm. The 226 height may be less than or equal to 5 mm.

The top width 222 may be less than or equal to 45 mm. The top width 222 may be less than or equal to 40 mm. The top width 222 may be less than or equal to 35 mm. The top width 222 may be less than or equal to 30 mm. The top width 222 may be less than or equal to 25 mm. The top width 222 may be less than or equal to 20 mm. The top width 222 may be less than or equal to 15 mm. The top width 222 may be less than or equal to 10 mm. The top width 222 may be less than or equal to 5 mm.

The top width 222 may be greater than or equal to 5 mm. The top width 222 may be greater than or equal to 10 mm. The top width 222 may be greater than or equal to 15 mm. The top width 222 may be greater than or equal to 20 mm. The top width 222 may be greater than or equal to 25 mm. The top width 222 may be greater than or equal to 30 mm. The top width 222 may be greater than or equal to 35 mm. The top width 222 may be greater than or equal to 40 mm.

The bottom width 224 may be less than or equal to 45 mm. The bottom width 224 may be less than or equal to 40 mm. The bottom width 224 may be less than or equal to 35 mm. The bottom width 224 may be less than or equal to 30 mm. The bottom width 224 may be less than or equal to 25 mm. The bottom width 224 may be less than or equal to 20 mm. The bottom width 224 may be less than or equal to 15 mm. The bottom width 224 may be less than or equal to 10 mm. The bottom width 224 may be less than or equal to 5 mm.

The bottom width 224 may be greater than or equal to 5 mm. The bottom width 224 may be greater than or equal to 10 mm. The bottom width 224 may be greater than or equal to 15 mm. The bottom width 224 may be greater than or equal to 20 mm. The bottom width 224 may be greater than or equal to 25 mm. The bottom width 224 may be greater than or equal to 30 mm. The bottom width 224 may be greater than or equal to 35 mm. The bottom width 224 may be greater than or equal to 40 mm.

As shown, the retractor 200 includes a groove 230. The groove 230 is configured to receive an indicator. The indicator may be radiolucent (e.g., if the body 220 is radiopaque) or radiopaque (e.g., if the body 220 is radiolucent) such that the location and orientation of the retractor 200 can be identified in an image (e.g., an x-ray).

According to other embodiments, the indicator is integrally formed in the body 220. For example, the body 220 may be formed of a plastic material that is over-molded onto an indicator (e.g., a radiolucent wire).

According to various embodiments, the retractor 200 includes metal material. Further, the retractor 200 may include radiolucent and/or radiopaque plastic material. The retractor 200 may be disposable. Alternatively, the retractor 200 may be formed of a material that can be sanitized and used again.

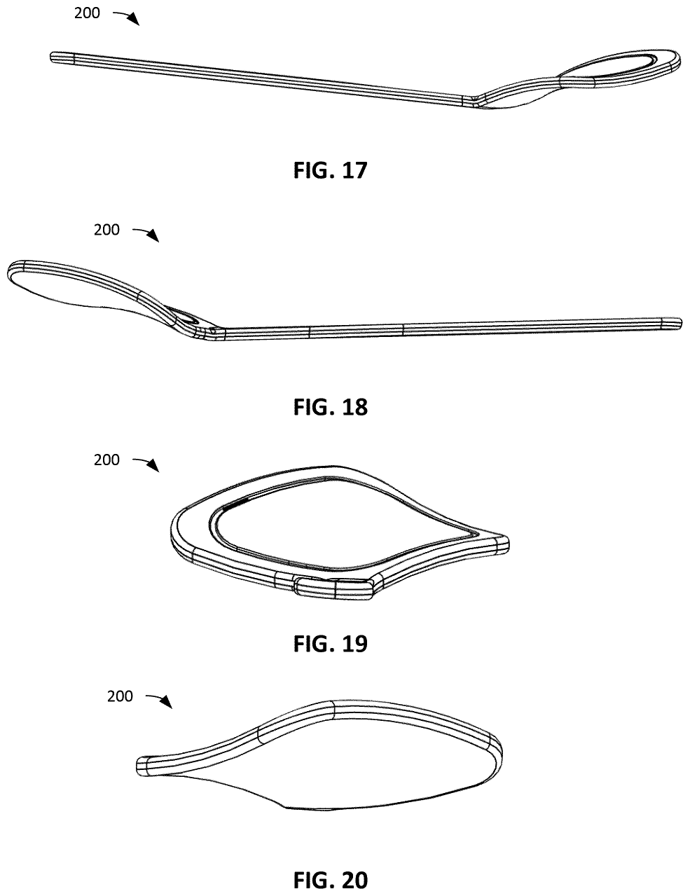

As best shown in FIGS. 17-20, the body 120 of the retractor 200 is curved. For example, the body 120 is shown to be curved in two planes. The curvature may correspond with the curvature of the ankle joint, as shown in FIGS. 11-14. According to various embodiments, the retractor 200 may be intended for use in the left ankle joint because of the curvature (e.g., as shown in FIGS. 11-14).

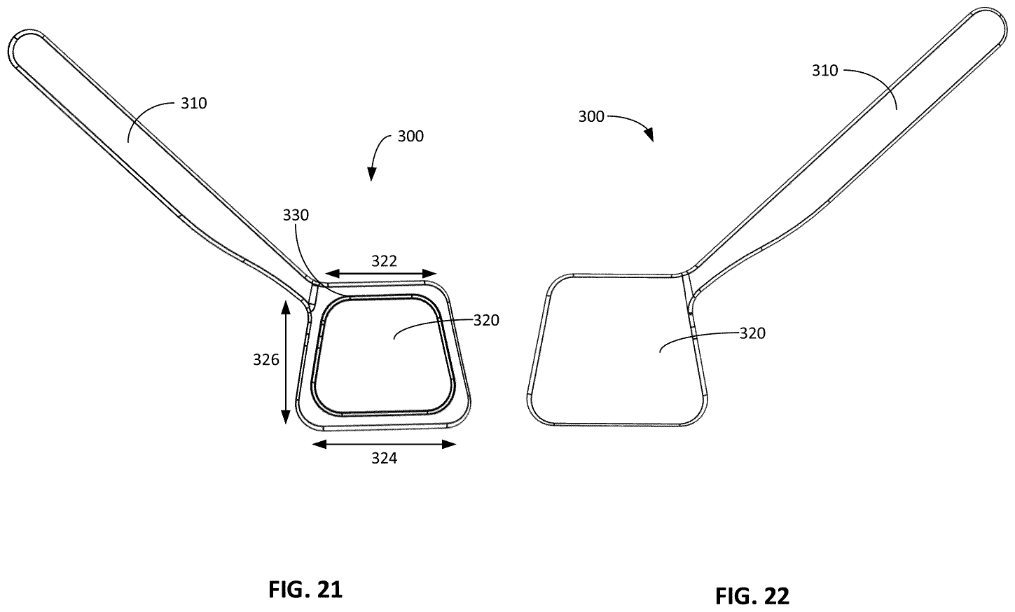

Referring now to FIGS. 21-26, another retractor 300 is shown according to an example embodiment. As shown, the retractor 300 includes a handle 310 coupled to a body 320. According to various embodiments, the handle 310 is integrally formed with the body 320. According to other embodiments, the handle 310 is removably coupled to the body 320 (e.g., via a clip, threads, snap feature, etc.).

The handle 310 is shown coupled to an upper corner of the body 320, however, according to other embodiments, the location of the handle 310 may be located along other locations of the top and lateral edge of the body 320. According to various embodiments, the position of the handle 310 relative to the body 320 may allow the retractor to be inserted into the desired location of the patient without the need to cut any tendons.

As shown, the body 320 defines a height 326, a top width 322 and a bottom width 324. The height 326, the top width 322, and the bottom width 324 may be adapted for use with any number of cutting jigs. According to various embodiments, the retractor 300 may be included in a kit of retractors, reaching being sized differently such that retractor 300 selected is chosen based on the size of the patient's ankle joint. According to various embodiments, the height 326 is less than or equal to 50 mm.

The 326 height may be less than or equal to 45 mm. The 326 height may be less than or equal to 40 mm. The 326 height may be less than or equal to 35 mm. The 326 height may be less than or equal to 30 mm. The 326 height may be less than or equal to 25 mm. The 326 height may be less than or equal to 20 mm. The 326 height may be less than or equal to 15 mm. The 326 height may be less than or equal to 10 mm. The 326 height may be less than or equal to 5 mm.

The top width 322 may be greater than or equal to 5 mm. The top width 322 may be greater than or equal to 10 mm. The top width 322 may be greater than or equal to 15 mm. The top width 322 may be greater than or equal to 20 mm. The top width 322 may be greater than or equal to 25 mm. The top width 322 may be greater than or equal to 30 mm. The top width 322 may be greater than or equal to 35 mm. The top width 322 may be greater than or equal to 40 mm.

The top width 322 may be less than or equal to 45 mm. The top width 322 may be less than or equal to 40 mm. The top width 322 may be less than or equal to 35 mm. The top width 322 may be less than or equal to 30 mm. The top width 322 may be less than or equal to 25 mm. The top width 322 may be less than or equal to 20 mm. The top width 322 may be less than or equal to 15 mm. The top width 322 may be less than or equal to 10 mm. The top width 322 may be less than or equal to 5 mm.

The top width 322 may be greater than or equal to 5 mm. The top width 322 may be greater than or equal to 10 mm. The top width 322 may be greater than or equal to 15 mm. The top width 322 may be greater than or equal to 20 mm. The top width 322 may be greater than or equal to 25 mm. The top width 322 may be greater than or equal to 30 mm. The top width 322 may be greater than or equal to 35 mm. The top width 322 may be greater than or equal to 40 mm.

The bottom width 324 may be less than or equal to 45 mm. The bottom width 324 may be less than or equal to 40 mm. The bottom width 324 may be less than or equal to 35 mm. The bottom width 324 may be less than or equal to 30 mm. The bottom width 324 may be less than or equal to 25 mm. The bottom width 324 may be less than or equal to 20 mm. The bottom width 324 may be less than or equal to 15 mm. The bottom width 324 may be less than or equal to 10 mm. The bottom width 324 may be less than or equal to 5 mm.

The bottom width 324 may be greater than or equal to 5 mm. The bottom width 324 may be greater than or equal to 10 mm. The bottom width 324 may be greater than or equal to 15 mm. The bottom width 324 may be greater than or equal to 20 mm. The bottom width 324 may be greater than or equal to 25 mm. The bottom width 324 may be greater than or equal to 30 mm. The bottom width 324 may be greater than or equal to 35 mm. The bottom width 324 may be greater than or equal to 40 mm.

As shown, the retractor 300 includes a groove 330. The groove 330 is configured to receive an indicator. The indicator may be radiolucent (e.g., if the body 320 is radiopaque) or radiopaque (e.g., if the body 320 is radiolucent) such that the location and orientation of the retractor 300 can be identified in an image (e.g., an x-ray).

According to other embodiments, the indicator is integrally formed in the body 320. For example, the body 320 may be formed of a plastic material that is over-molded onto an indicator (e.g., a radiolucent wire).

According to various embodiments, the retractor 300 includes metal material. Further, the retractor 300 may include radiolucent and/or radiopaque plastic material. The retractor 300 may be disposable. Alternatively, the retractor 300 may be formed of a material that can be sanitized and used again.

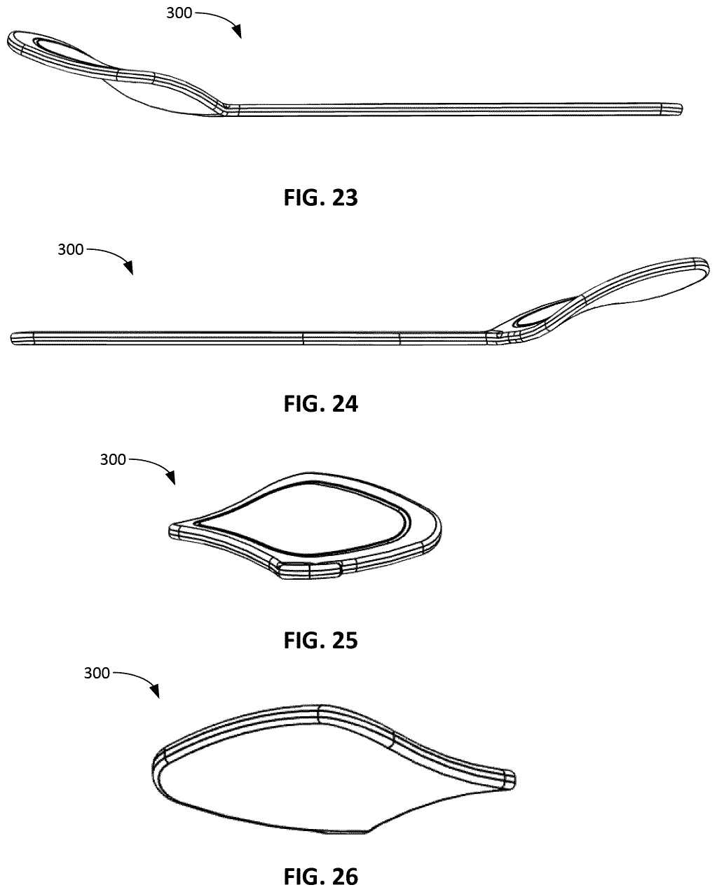

As best shown in FIGS. 23-26, the body 120 of the retractor 200 is curved. For example, the body 120 is shown to be curved in two planes. The curvature may correspond with the curvature of the ankle joint. According to various embodiments, the retractor 200 may be intended for use in the right ankle joint because of the curvature.

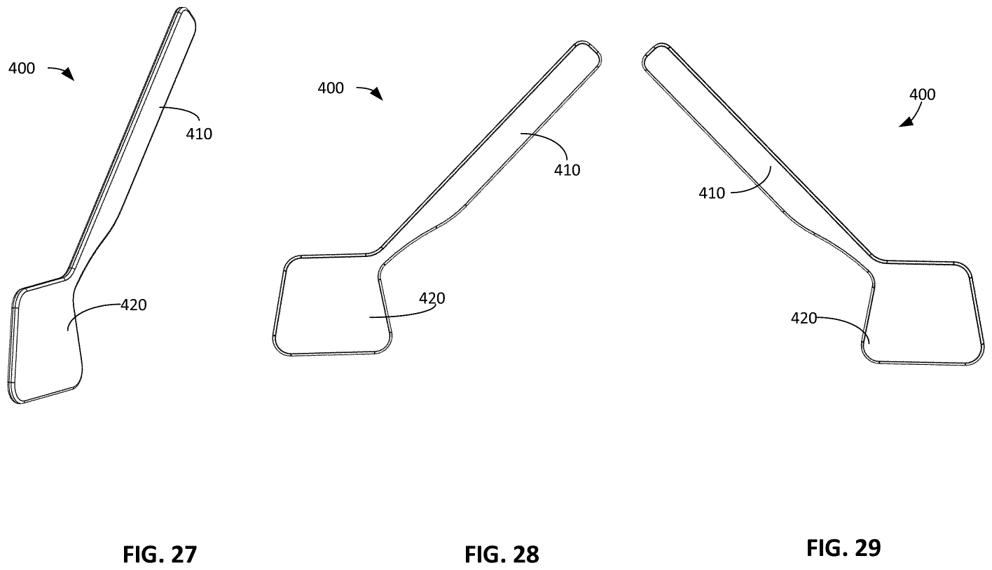

Referring now to FIGS. 27-29, another retractor 400 is shown, according to an example embodiment. The retractor 400 includes a handle 410 coupled to a body 420. The retractor 400 may share one or more features with the retractor 100 described above. However, the retractor 400 does not include a groove.

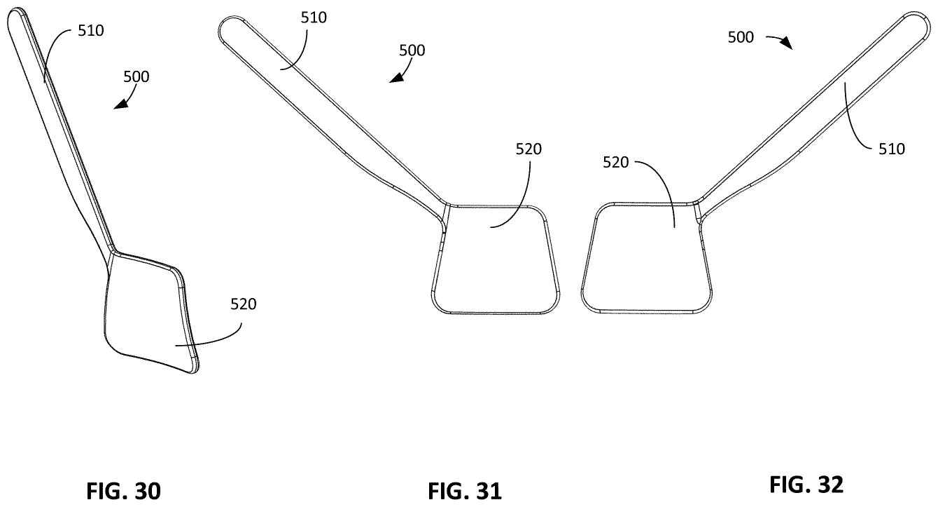

Referring now to FIGS. 30-32, another retractor 500 is shown, according to an example embodiment. The retractor 500 includes a handle 510 coupled to a body 520. The retractor 500 may share one or more features with the retractor 200 described above. However, the retractor 500 does not include a groove.

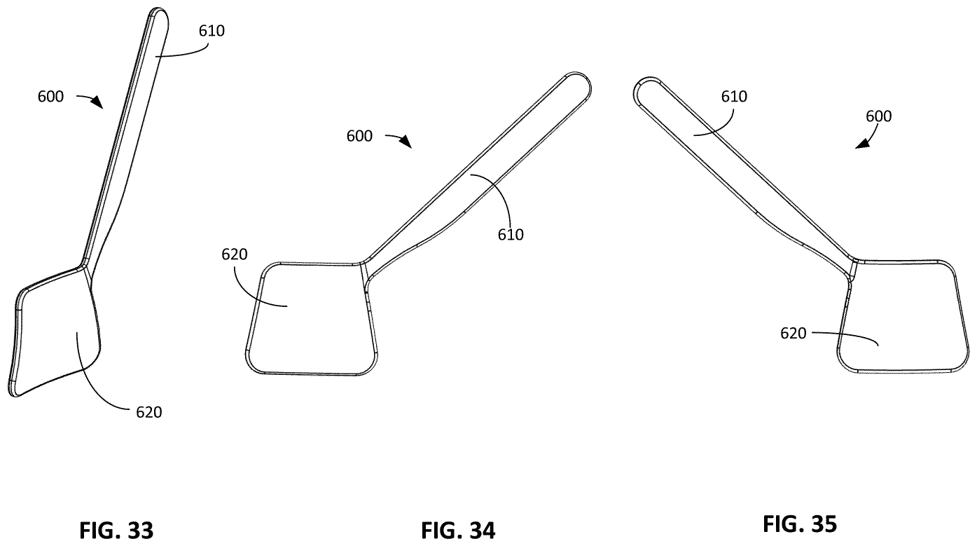

Referring now to FIGS. 33-35, another retractor 600 is shown, according to an example embodiment. The retractor 600 includes a handle 610 coupled to a body 620. The retractor 600 may share one or more features with the retractor 300 described above. However, the retractor 600 does not include a groove.

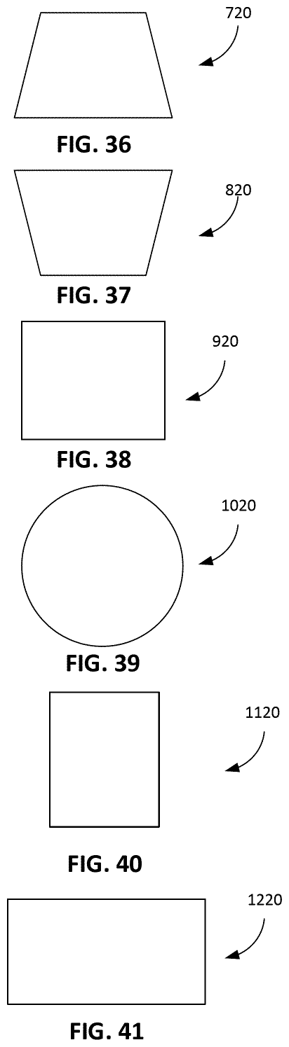

Referring now to FIGS. 36-41, bodies 720, 820, 920, 1020, 1120, 1220 are shown, according to example embodiments. The bodies 720, 820, 920, 1020, 1120, 1220 may be coupled to a handle as a part of a retractor. As shown, the bodies of the retractors described herein may be a number of different shapes, such as a trapezoid, a square, a circle, an oval, a rectangle, or an abstract shape. It should be appreciated that each of the bodies 720, 820, 920, 1020, 1120, 1220 may be flat, curved in one plane, curved in two planes, etc.

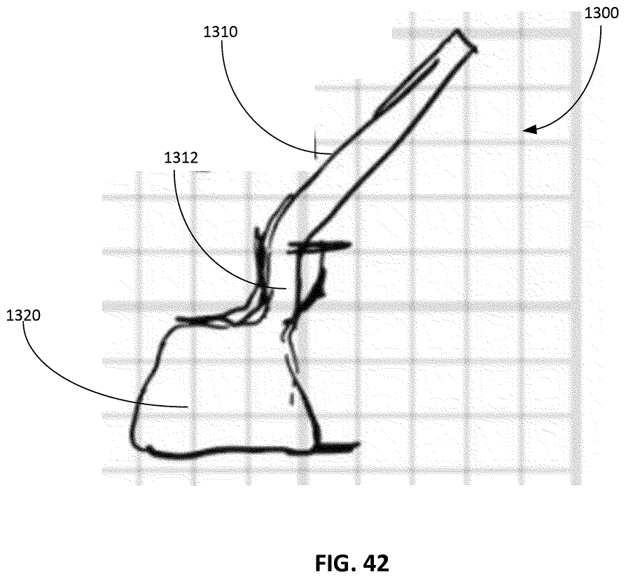

Referring now to FIG. 42, a retractor 1300 is shown, according to an example embodiment. As shown, the retractor 1300 includes a handle 1310 coupled to a body 1320. As shown, the handle 1310 includes an extended neck 1312, which may facilitate positioning the retractor in the desired location.

A retractor (e.g., retractor 1400, retractor 1600) of the present disclosure includes components described with reference to an X, Y, Z, coordinate plane. An X-direction may be a lateral direction (e.g., a direction extending between side edge portions of the retractor, a direction extending between medial and lateral sides, a direction extending between left and right sides etc.). A Y-direction may be a vertical direction (e.g., a direction extending between a top edge section and a bottom edge section of the retractor, a direction extending between superior and inferior sides, etc.). A Z-direction may be a longitudinal direction (e.g., a direction extending between an inner surface and an outer surface of the retractor, a direction extending between posterior and anterior sides, etc.).

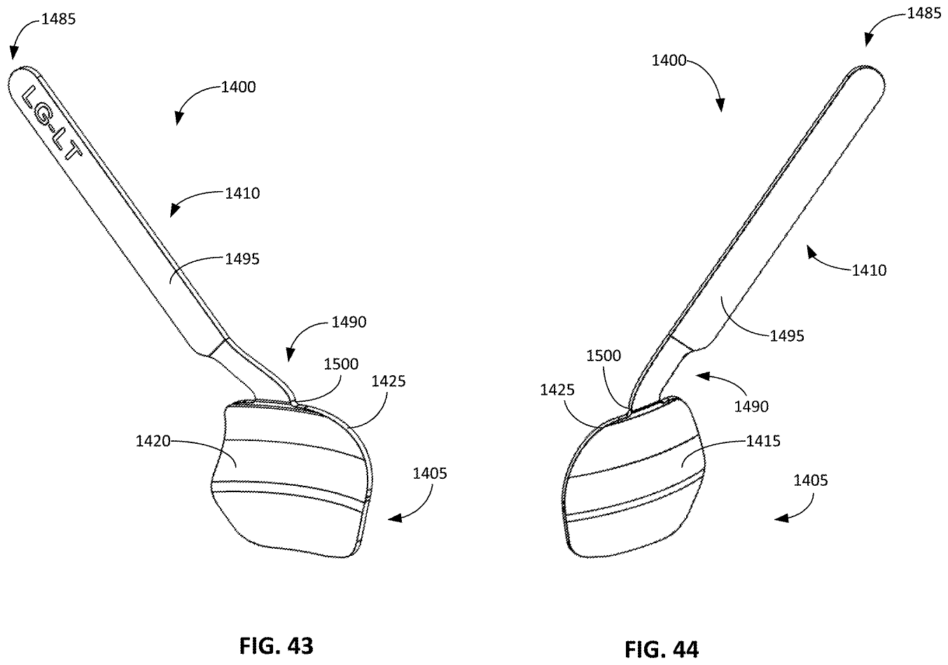

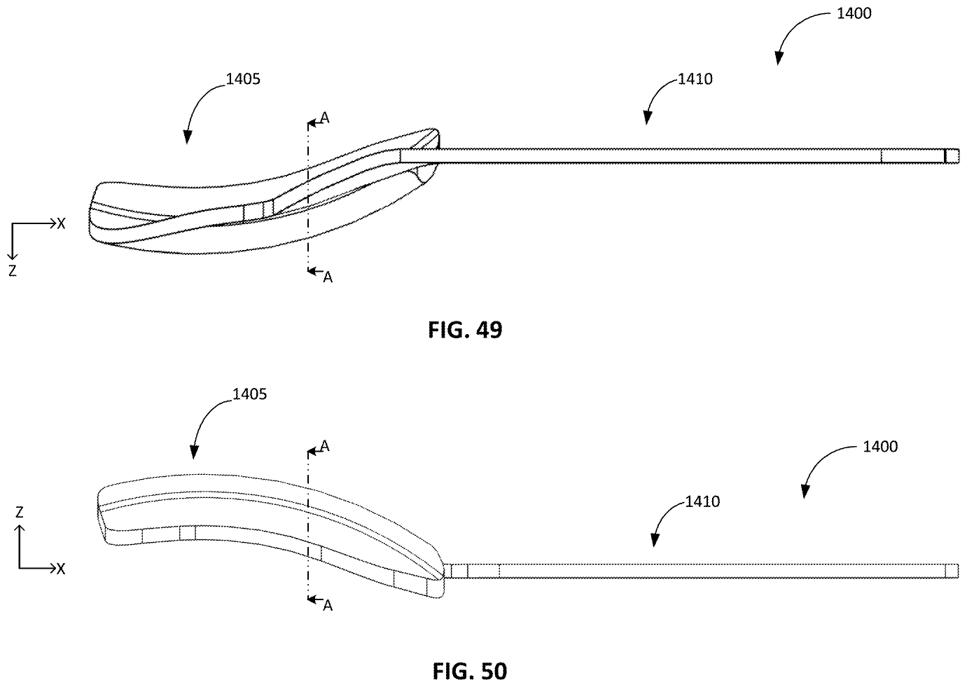

Referring now to FIGS. 43-52, a retractor 1400 (e.g., a surgical retractor device) is shown, according to an example embodiment. The retractor 1400 includes a body 1405 and a handle 1410 extending from a portion of the body 1405. As shown, the body 1405 and the handle 1410 are integrally formed with each other to form a single unitary body. In some examples, the body 1405 and the handle 1410 are detachable from each other. According to various examples, the retractor 1400 may be inserted into a patient (e.g., inserted into the patient via an incision) in a surgical procedure. The retractor 1400 may be inserted into the patient such that the body 1405 is positioned inside the patient and the handle 1410 extends out of the patient via the incision. The handle 1410 may be gripped by a user (e.g., a surgeon, a nurse, etc.) to position the body 1405 within the patient to a desired position. Generally, the retractor 1400 is positioned to prevent a cutting jig from traveling beyond an undesired location within the patient, thereby protecting tissue, nerves, tendons (e.g., achilleas tendon), vascular structures, etc. of the patient.

In some examples, the retractor 1400 (e.g., the body 1405 and/or the handle 1410) is radiopaque such that a location and orientation of the retractor 1400 can be identified in an image (e.g., an x-ray). In such examples, the body 1405 may be made from a radiopaque material such as aluminum, stainless steel, or another suitable material.

As shown, the body 1405 includes an outer surface 1415, an inner surface 1420 opposite the outer surface 1415, and a peripheral edge 1425. The body 1405 defines one or more curved profiles such that the outer surface 1415 is convex and the inner surface 1420 is concave.

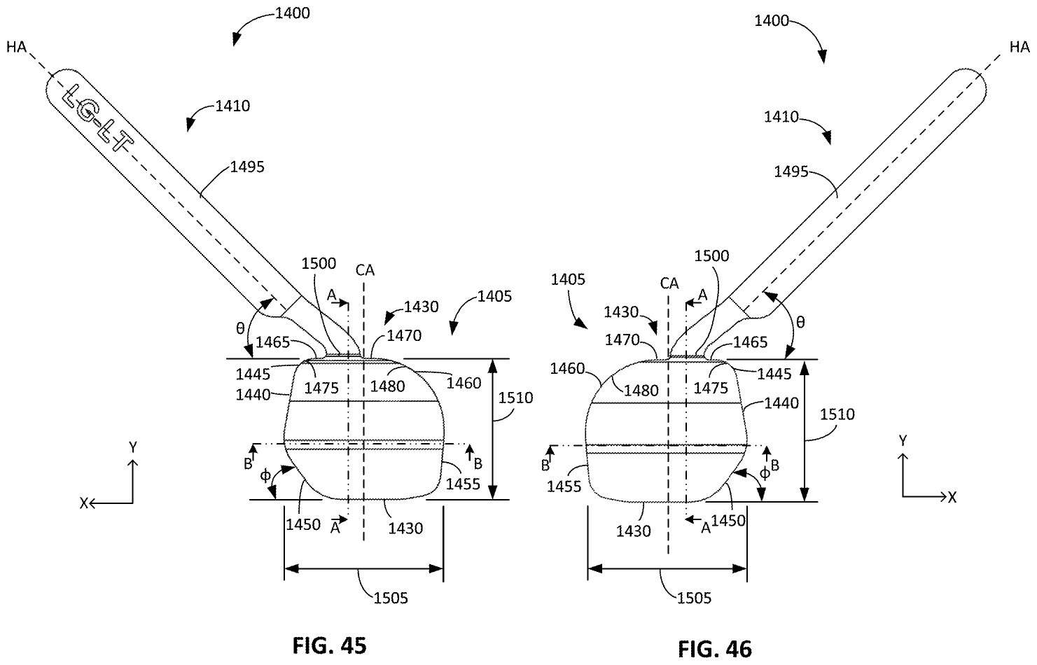

Referring to FIGS. 45 and 46, the peripheral edge 1425 includes a top edge section 1430, a bottom edge section 1435, a first side edge portion 1440, a first transition edge portion 1445, an angled edge portion 1450, a second side edge portion 1455, and a second transition edge portion 1460. The top edge section 1430 includes a first top edge portion 1465 and a second top edge portion 1470. The bottom edge section 1435 is spaced from the top edge section 1430 (e.g., vertically offset from the top edge section 1430 in a Y-direction) and is substantially parallel (e.g., within 5% of parallel, within 10% of parallel, etc.) with the top edge section 1430.

The first side edge portion 1440 of the peripheral edge 1425 is substantially perpendicular (e.g., within 5% of perpendicular, within 10% of perpendicular, etc.) to the top edge section 1430 (e.g., to the first top edge portion 1465 of the top edge section 1430) and to the bottom edge section 1435.

As shown in FIGS. 45 and 46, the first transition edge portion 1445 is between the first top edge portion 1465 and the first side edge portion 1440. For example, the first transition edge portion 1445 connects the first top edge portion 1465 with the first side edge portion 1440 along the peripheral edge 1425. The first transition edge portion 1445 may be rounded, beveled, filleted, etc., such that the first transition edge portion 1445 defines a radius or curvature, shown as first radius 1475.

As shown, the angled edge portion 1450 of the peripheral edge 1425 extends between the first side edge portion 1440 and the bottom edge section 1435. The angled edge portion 1450 extends at an angle, shown as angle φ, between the first side edge portion 1440 and the bottom edge section 1435. The angle φ may be an angle defined within an X-Y plane between the angled edge portion 1450 and the bottom edge section 1435. The angle φ may be about 45° (e.g., about 40°, about 50°, etc.). In some examples, the angle φ is greater or less than 45° (e.g., about 30°, about 20°, about 60°, about 75°, etc.).

The second side edge portion 1455 spaced from the first side edge portion 1440 (e.g., laterally offset from the first side edge portion 1440 in the X-direction) and is substantially parallel (e.g., within 5% of parallel, within 10% of parallel, etc.) with the first side edge portion 1440. The second side edge portion 1455 is substantially perpendicular (e.g., within 5% of perpendicular, within 10% of perpendicular, etc.) to the top edge section 1430 (e.g., to the second top edge portion 1470 of the top edge section 1430) and to the bottom edge section 1435. In some examples, the second side edge portion 1455 extends directly from the bottom edge section 1435. In other examples, a transition portion such as a rounded, beveled, filleted, angled, etc., portion is between the second side edge portion 1455 and the bottom edge section 1435. In such examples, the transition portion connects the second side edge portion 1455 with the bottom edge section 1435.

As shown, the second transition edge portion 1460 is between the second top edge portion 1470 and the second side edge portion 1455. For example, the second transition edge portion 1460 connects the second top edge portion 1470 with the second side edge portion 1455 along the peripheral edge 1425. The second transition edge portion 1460 may be rounded, beveled, filleted, etc., such that the second transition edge portion 1460 defines a radius or curvature, shown as second radius 1480. As shown, the second radius 1480 of the second transition edge portion 1460 is larger than the first radius 1475 of the first transition edge portion 1445.

As shown in FIGS. 45 and 46, the handle 1410 defines a proximal end 1485 and a distal end 1490 opposite the proximal end 1485. The handle 1410 extends from the peripheral edge 1425 of the body 1405. For example, the distal end 1490 connects to or otherwise extends from the body 1405. As shown, the handle 1410 extends from a portion of the peripheral edge 1425 between (e.g., laterally between) the first top edge portion 1465 and the second top edge portion 1470 of the top edge section 1430. In some examples, the handle 1410 extends from a different portion of the body 1405. In such examples, the handle 1410 may extend from a different position along the peripheral edge 1425. For example, the handle 1410 may extend from at least one of the top edge section 1430, the first transition edge portion 1445, or the first side edge portion 1440.

As shown, the handle 1410 extends from the peripheral edge 1425 at an angle, shown as angle θ, relative to the body 1405. The angle θ may be an angle defined within an X-Y plane between the first top edge portion 1465 and a center axis of the handle 1410, shown as handle axis HA. The angle θ may be about 45° (e.g., about 40°, about 50°, etc.). In some examples, the angle θ is greater or less than 45° (e.g., about 30°, about 20°, about 60°, about 75°, etc.).

In some examples, the handle 1410 includes a first handle portion 1495 and a second handle portion 1500 extending at an angle from the first handle portion 1495. As shown, the second handle portion 1500 extends from at least one of the top edge section 1430, the first transition edge portion 1445, or the first side edge portion 1440. In some examples, the second handle portion 1500 extends substantially perpendicularly (e.g., within 5% of perpendicular, within 10% of perpendicular, etc.) from the peripheral edge 1425 of the body 1405 such that the angle at which the second handle portion 1500 extends from the first handle portion 1495 is about equal to a difference between the angle θ and about 90°. For example, if the angle θ is about 45°, the angle at which the second handle portion 1500 extends from the first handle portion 1495 is about 45°. Additionally or alternatively, the second handle portion 1500 extends from the first handle portion 1495 at an angle defined within a Y-Z plane such that the second handle portion 1500 and the first handle portion 1495 are longitudinally offset from each other.

As shown, the body 1405 defines a width 1505. The width 1505 may be a lateral distance between the first side edge portion 1440 and the second side edge portion 1455 (e.g., a maximum lateral distance between the first side edge portion 1440 and the second side edge portion 1455). For example, the width 1505 may be a lateral distance between (i) a point where the first side edge portion 1440 meets the angled edge portion 1450 and (ii) the second side edge portion 1455 of the peripheral edge 1425 of the body 1405. The width 1505 may be between about 20 mm and about 60 mm. In some examples, the width 1505 is otherwise suitably dimensioned (e.g., greater or less than 20 mm, greater or less than 60 mm, etc.).

As shown, the body 1405 defines a height 1510. The height 1510 may be a vertical distance between the top edge section 1430 and the bottom edge section 1435 (e.g., a maximum vertical distance between the top edge section 1430 and the bottom edge section 1435). For example, the height 1510 may be a vertical distance between (i) the first top edge portion 1465 or the second top edge portion 1470 and (ii) the bottom edge section 1435 of the peripheral edge 1425 of the body 1405. The height 1510 may be between about 20 mm and about 60 mm. In some examples, the height 1510 is otherwise suitably dimensioned (e.g., greater or less than 20 mm, greater or less than 40 mm, etc.).

As shown in FIGS. 45 and 46, the body 1405 defines a central axis CA. The central axis CA may be an axis extending in a vertical direction (e.g., a Y-axis) and located at a lateral midpoint of the width 1505 of the body 1405. The body 1405 is shaped (e.g., profiled as viewed from FIGS. 45 and 46) such that first and second sections (e.g., lateral and medial sections, left and right sections, etc.) of the body 1405 are asymmetrical across the central axis CA. In other words, the shape of the peripheral edge 1425 is not mirrored across the central axis CA (e.g., the first side edge portion 1440 defines a profile that is different than a profile defined by the second side edge portion 1455).

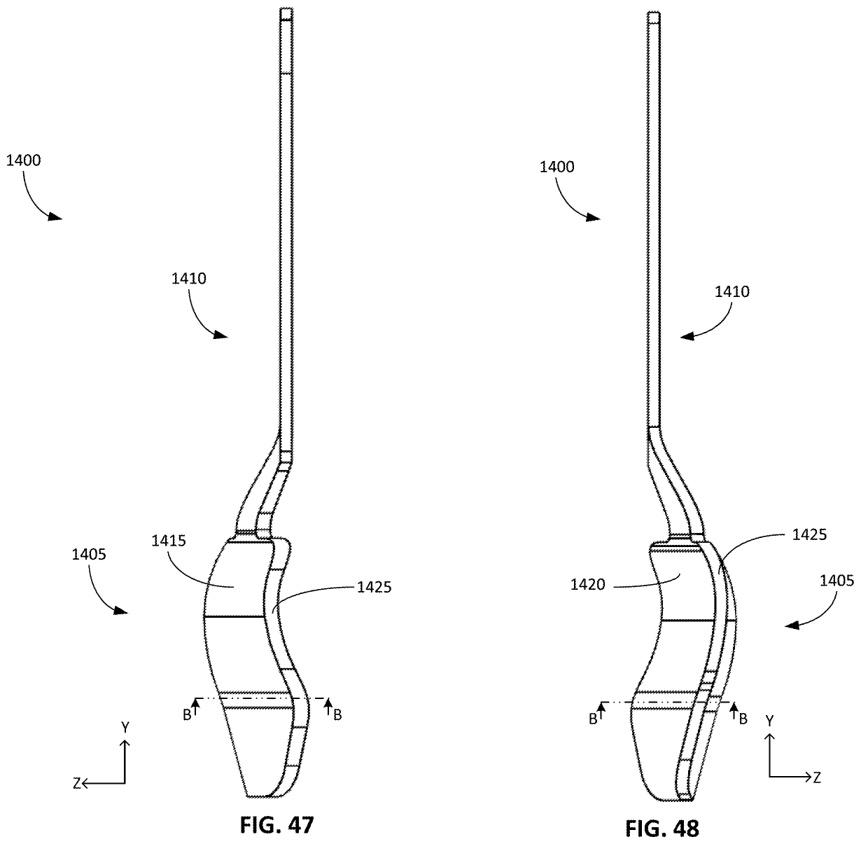

Referring now to FIGS. 47-50, the first side edge portion 1440 and/or the angled edge portion 1450 are longitudinally offset from the second side edge portion 1455 and/or the second transition edge portion 1460.

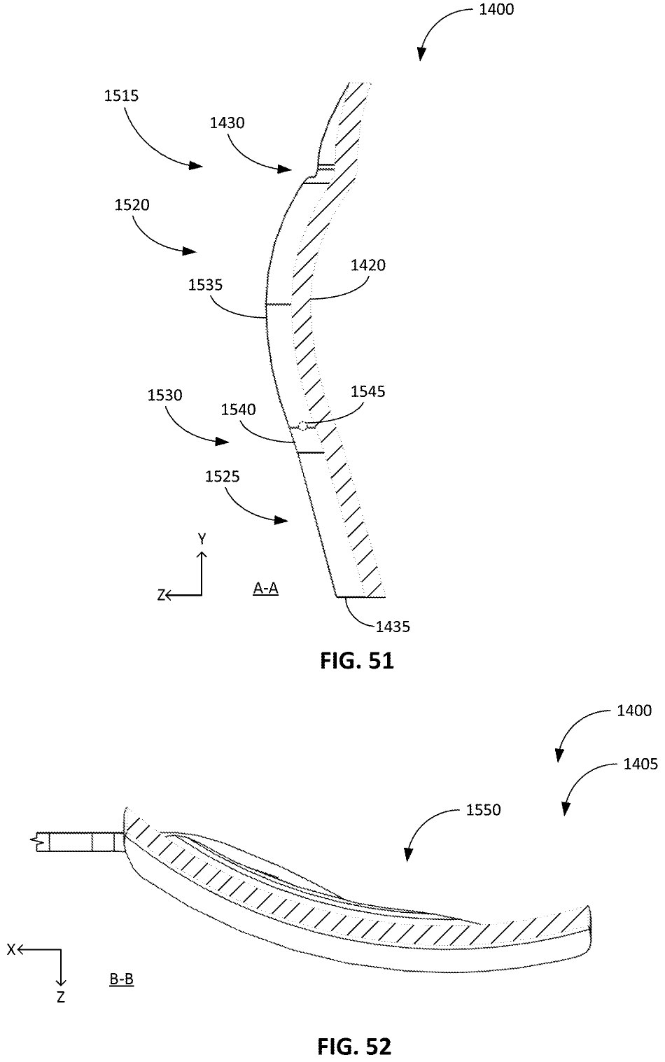

Referring now to FIG. 51, a cross-sectional view of the body 1405 is shown within a plane A-A (e.g., a first plane). The plane A-A may be a Y-Z plane positioned proximate a lateral midpoint of the width 1505. In some examples, the plane A-A is otherwise laterally positioned along the width 1505 of the body 1405. The plane A-A may be substantially parallel (e.g., within 5% of parallel, within 10% of parallel, etc.) with the central axis CA. In some examples, the central axis CA extends within the plane A-A.

As shown, the body 1405 defines a curved profile, shown as first profile 1515, within the plane A-A. The first profile 1515 includes a curved portion 1520, a flat portion 1525, and a transition portion 1530 between the curved portion 1520 and the flat portion 1525. The curved portion 1520 of the first profile 1515 defines a convex outer surface 1535 of the body 1405. The transition portion 1530 of the first profile 1515 defines a concave outer surface 1540 of the body 1405. The first profile 1515 includes an inflection point 1545 between the curved portion 1520 and the transition portion 1530 such that a direction of a curvature along the first profile 1515 changes (e.g., along a vertical Y-direction between the top edge section 1430 and the bottom edge section 1435).

As shown, the first profile 1515 extends within the A-A plane (e.g., in a vertical Y-direction as shown in FIG. 51) between the top edge section 1430 and the bottom edge section 1435. For example, the first profile 1515 extends within the A-A plane between (i) the first top edge portion 1465 and the second top edge portion 1470 and (ii) the bottom edge section 1435 of the peripheral edge 1425 of the body 1405.

Referring now to FIG. 52, a cross-sectional view of the body 1405 is shown within a plane B-B (e.g., a second plane). The plane B-B may be a X-Z plane positioned proximate a vertical midpoint of the height 1510. In some examples, the plane B-B is otherwise vertically positioned along the height 1510 of the body 1405. The plane B-B may be substantially perpendicular to the plane A-A (e.g., within 5% of perpendicular, within 10% of perpendicular, etc.).

As shown, the body 1405 defines a curved profile, shown as second profile 1550, within the plane B-B. The second profile 1550 extends within the plane B-B (e.g., in a lateral X-direction as shown in FIG. 52) between (i) the first side edge portion 1440 or the angled edge portion 1450 and (ii) the second transition edge portion 1460 or the second side edge portion 1455 of the peripheral edge 1425 of the body 1405.

As shown in FIG. 52, the outer surface 1415 of the body 1405 defined by the second profile 1550 is convex. The outer surface 1415 of the body 1405 defined by the second profile 1550 may be continuously convex (e.g., defining the same radius, having no inflection points, etc.) in the lateral X-direction. For example, the outer surface 1415 of the second profile 1550 may be continuously convex between (i) the first side edge portion 1440 or the angled edge portion 1450 and (ii) the second transition edge portion 1460 or the second side edge portion 1455 of the peripheral edge 1425 of the body 1405.

According to various embodiments, the retractor 1400 may be intended for use in a left ankle joint because of the orientation of the concavity defined by the body 1405 and the direction of extension of the handle 1410 from the body 1405.

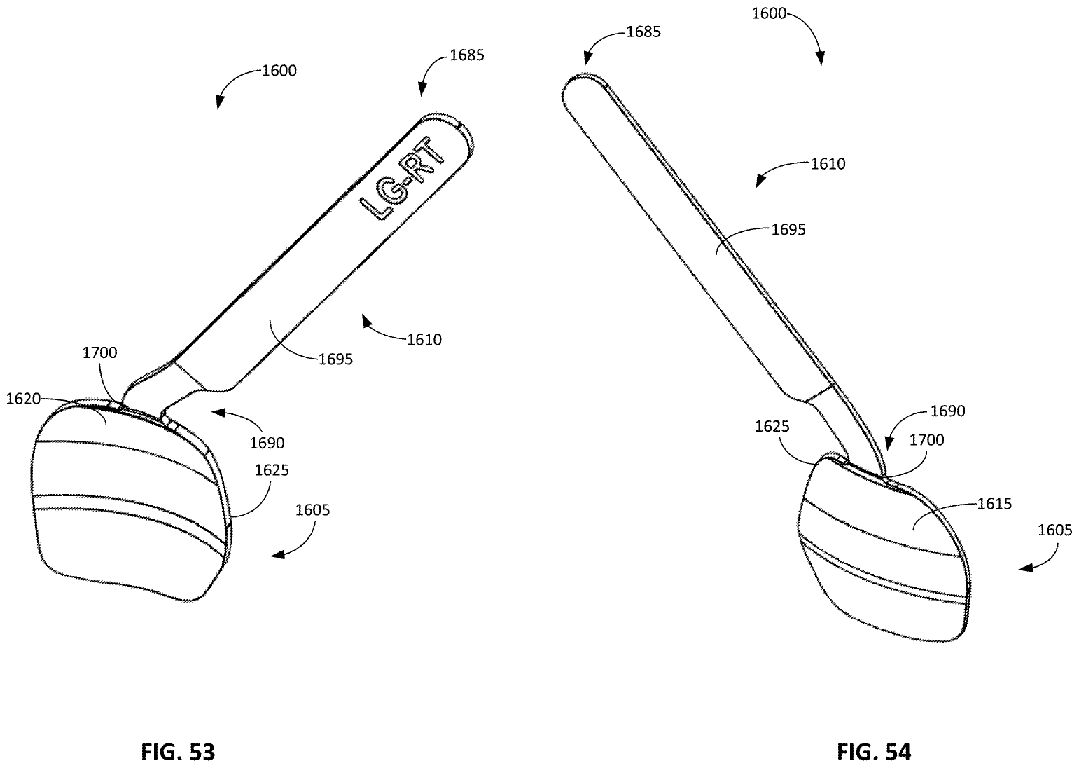

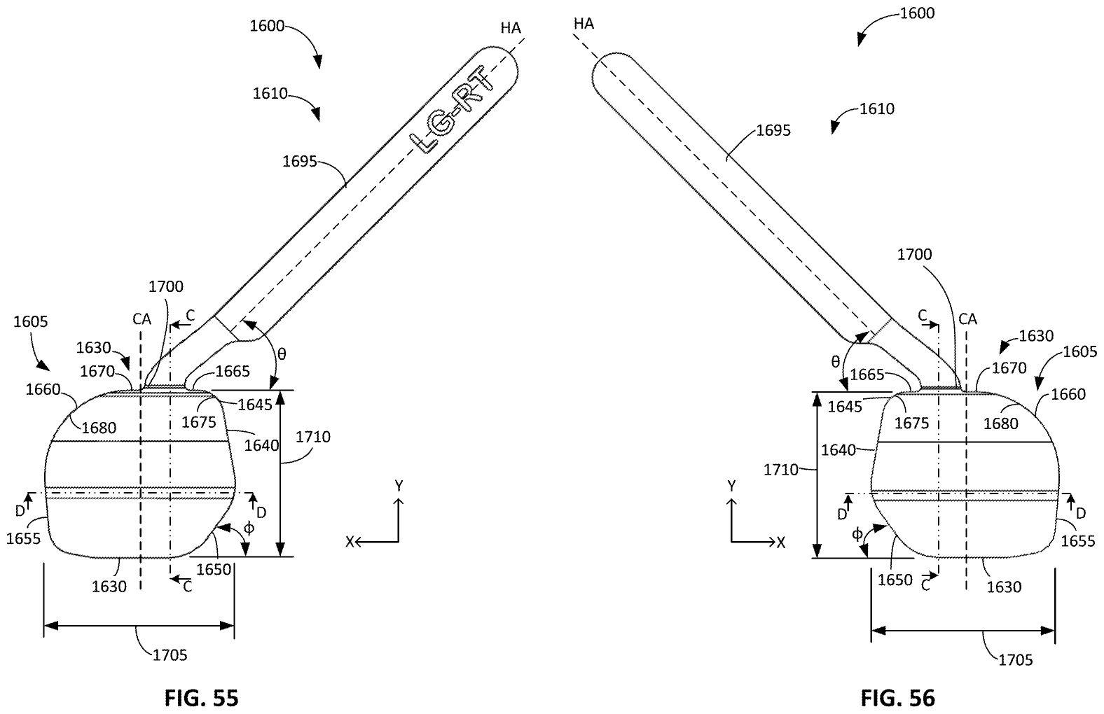

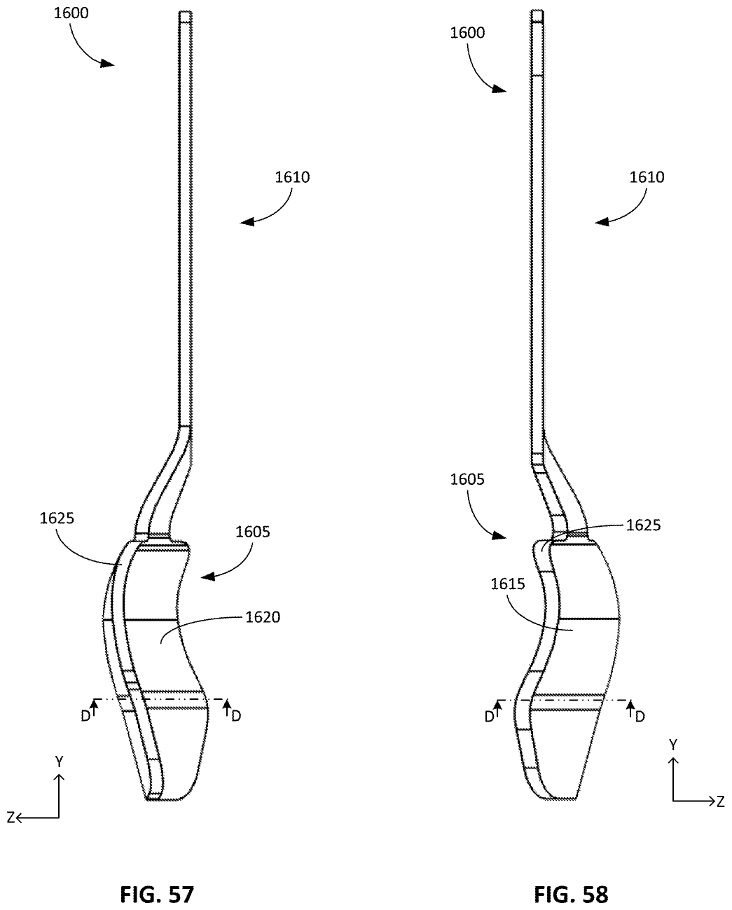

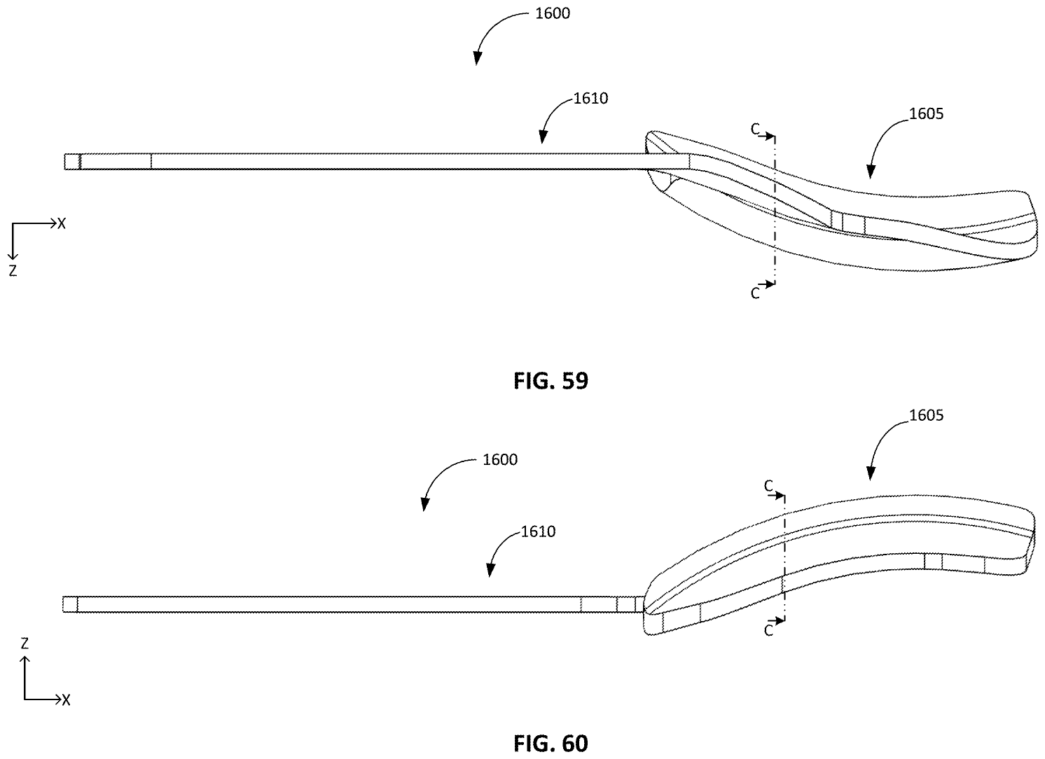

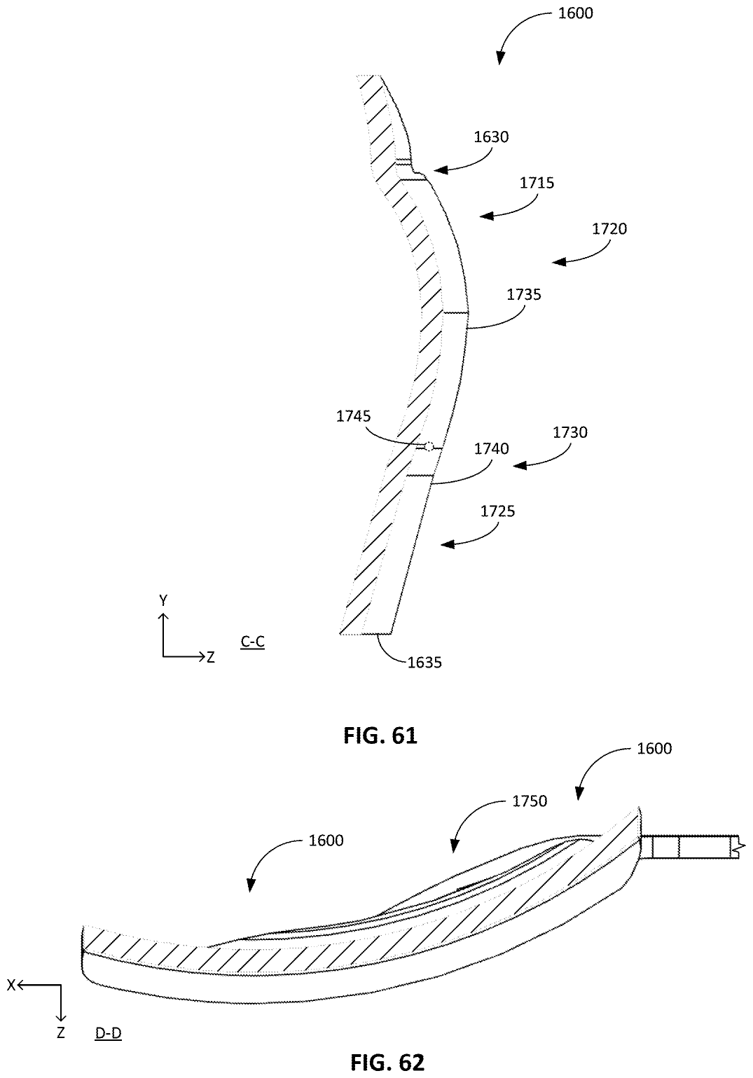

Referring now to FIGS. 53-63, another retractor 1600 is shown including a body 1605 and a handle 1610, according to an example embodiment. The retractor 1600 may share one or more features with the retractor 1400 described above. However, the retractor 1600 may define an orientation of a concavity defined by the body 1605 and a direction of extension of the handle 1610 from the body 1605 such that the retractor 1600 is intended for use in a right ankle joint. For example, the various components (and the relative positions and orientations thereof) of the retractor 1600 are substantially similar to the various components of the retractor 1400 and are mirrored (e.g., such that the retractor 1600 is intended for use in a right ankle joint as opposed to a left ankle joint).

As shown, the retractor 1600 includes the body 1605 defining an outer surface 1615, an inner surface 1620, a peripheral edge 1625 having a top edge section 1630, a bottom edge section 1635, a first side edge portion 1640, a first transition edge portion 1645 defining a first radius 1675, an angled edge portion 1650, a side edge portion 1655, a second transition edge portion 1660 defining a second radius 1680, a first top edge portion 1665, a second top edge portion 1670, an angle φ, a width 1705, and a height 1710.

As shown, the retractor 1600 includes the handle 1610 having a proximal end 1685, a distal end 1690, a first handle portion 1695, and a second handle portion 1700. The handle 1610 extending from the body 1605 at an angle θ along the handle axis HA.

As shown, the retractor 1600 defines a first profile 1715 within a plane C-C (e.g., a first plane). The first profile 1715 having a curved portion 1720 defining a convex outer surface 1735, a flat portion 1725, and a transition portion 1730 defining a concave outer surface 1740, and an inflection point 1745. The retractor 1600 defines a second profile 1750 within a plane D-D (e.g., a second plane).

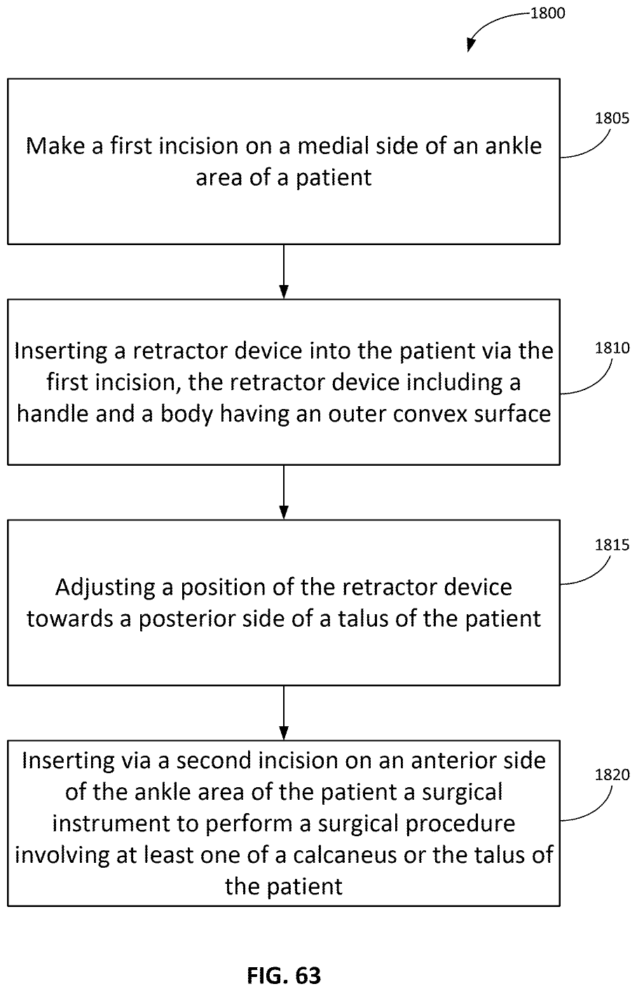

As shown in FIG. 63, a method of performing a surgery (e.g., a total ankle replacement, total ankle arthroplasty, ankle fusion, etc.), shown as method 1800, includes steps 1805-1820.

At step 1805, a first incision is made on a medial side of an ankle area of a patient. The first incision may be a vertical cut along the medial side of the ankle area of the patient. In some examples, the first incision is just long enough to facilitate inserting a retractor (e.g., any of the retractors discussed herein such as retractor 1400, retractor 1600, etc.) through the first incision. For example, the length of the first incision may be about equal to a height of the retractor.

At step 1810, a retractor device (e.g., any of the retractors discussed herein such as retractor 1400, retractor 1600, etc.) is inserted into the patient via the first incision. The retractor device includes a handle (e.g., handle 1410, handle 1610, etc.) and a body (e.g., body 1405, body 1605, etc.) having an outer convex surface. In some examples, the retractor device is inserted into the patient at an angle relative to the first incision. In some examples, the outer convex surface is structured to conform to a contour of the anatomy of the patient. For example, the outer convex surface is structured to conform to the shape of the posterior malleolus of the patient, the shape of the tibia, the shape of the fibula, etc.

At step 1815, a position of the retractor device is adjusted to be positioned towards a posterior side of a talus of the patient. In some examples, the position of the retractor device is adjusted to be positioned towards a posterior side of a calcaneus of the patient. The handle of the retractor device extends out of the patient through the incision such that a user (e.g., surgeon, nurse, etc.) can grip or otherwise engage with the handle. The handle and the body may be integrally formed with each other such that movement of the handle moves the body. The user may grip the handle and manipulate the position of the handle to position the body at a desired position and a desired orientation within the patient. An imaging device (e.g., x-ray device) may be used to properly position and orient the body of the retractor device.

At step 1820, a surgical instrument is inserted into the patient via a second incision on an anterior side of the ankle area of the patient. The surgical instrument is to perform a surgical procedure involving at least one of the calcaneus or the talus of the patient. The surgical instrument may be a drill, a saw, a reamer, cutting jig, or the like used to remove bone structure in the respective ankle (e.g., a portion of the tibia, a portion of the talus, a portion of the talus, etc.). At step 1815, the body of the retractor device is positioned and oriented to protect nearby tissues (e.g., muscle, tendons, arteries, etc.) and prevent the surgical instrument from cutting or removing the nearby tissue that is not intended to be cut by the surgical instrument during the surgery.

As utilized herein with respect to numerical ranges, the terms “approximately,” “about,” “substantially,” and similar terms generally mean+/−10% of the disclosed values, unless specified otherwise. As utilized herein with respect to structural features (e.g., to describe shape, size, orientation, direction, relative position, etc.), the terms “approximately,” “about,” “substantially,” and similar terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References herein to the positions of elements (e.g., “top,” “bottom,” “above,” “below”) are merely used to describe the orientation of various elements in the figures. It should be noted that the orientation of various elements may differ according to other exemplary embodiments, and that such variations are intended to be encompassed by the present disclosure.

Although the embodiments are numbered with, for example, “first,” “second,” or “third,” or “fourth,” the ordinal numbers do not imply priorities of the embodiments.

The use of “e.g.,” “etc.,” “for instance,” “in example,” and “or” and grammatically related terms indicates non-exclusive alternatives without limitation, unless otherwise noted. The use of “optionally” and grammatically related terms means that the subsequently described element, event, feature, or circumstance may or may not be present/occur, and that the description includes instances where said element, event, feature, or circumstance occurs and instances where it does not. The use of “exemplary” refers to “an example of” and is not intended to convey a meaning of an ideal or preferred embodiment. The use of “attached” and “coupled” grammatically related terms refers to the fixed, releasable, or integrated association of two or more elements and/or devices with or without one or more other elements in between. Thus, the term “attached” or “coupled” and grammatically related terms includes releasably attaching or fixedly attaching two or more elements and/or devices in the present or absence of one or more other elements in between. As used herein, the terms “proximal” and “distal” are used to describe opposing axial ends of the particular elements or features being described in relation to anatomical placement. As used herein, the terms “proximal,” “distal,” “inferior,” “posterior,” and any other relative position terms are intended to facilitate clarity regarding the disclosed embodiments, and do not limit the disclosure to any particular frame of reference.

Although the figures and descriptions may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It is important to note that the construction and arrangement of the retractors (e.g., retractor 1400, retractor 1600, etc.) and the systems and components thereof (e.g., the body 1405, the body 1605, the handle 1410, the handle 1610, etc.) as shown in the various exemplary embodiments is illustrative only. Additionally, any element disclosed in one embodiment may be incorporated or utilized with any other embodiment disclosed herein.

Since many modifications, variations and changes in detail can be made to the described embodiments of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A surgical retractor device, comprising:
a body having one or more curved profiles with an outer convex surface and including a peripheral edge, wherein the peripheral edge of the body includes:
a top edge section of the peripheral edge including a first top edge portion and a second top edge portion;
a first side edge portion substantially perpendicular to the first top edge portion;
a first transition edge portion between the first top edge portion and the first side edge portion, the first transition edge portion defining a first radius;
a bottom edge section;
an angled edge portion extending at an angle between the first side edge portion and the bottom edge section;
a second side edge portion opposite the first side edge portion and substantially perpendicular to the second top edge portion; and
a second transition edge portion between the second top edge portion and the second side edge portion, the second transition edge portion defining a second radius,
wherein the second radius of the second transition edge portion is larger than the first radius of the first transition edge portion; and
a handle having a proximal end and a distal end, the distal end of the handle extending from at least one of the top edge section, the first transition edge portion, or the first side edge portion.

2. The surgical retractor device of claim 1, wherein the handle extends from the at least one of the top edge section, the first transition edge portion, or the first side edge portion at an angle relative to the body.

3. The surgical retractor device of claim 1, wherein the handle includes a first handle portion and a second handle portion extending at an angle from the first handle portion, the second handle portion extending from at least one of the top edge section, the first transition edge portion, or the first side edge portion.

4. The surgical retractor device of claim 1, wherein the body is asymmetrical across a central axis.

5. The surgical retractor device of claim 1, wherein the one or more curved profiles defined by the body include a first profile within a first plane and a second profile in a second plane perpendicular to the first plane.

6. The surgical retractor device of claim 5, wherein the first profile includes a curved portion, a flat portion, and a transition portion.

7. The surgical retractor device of claim 6, wherein the curved portion of the first profile defines a convex outer surface of the body.

8. The surgical retractor device of claim 7, wherein the transition portion of the first profile defines a concave outer surface of the body.

9. The surgical retractor device of claim 8, wherein the first profile extends within the first plane between (i) the first top edge portion and the second top edge portion and (ii) the bottom edge section of the peripheral edge of the body.

10. The surgical retractor device of claim 6, wherein the first profile includes an inflection point between the curved portion and the transition portion such that a direction of a curvature along the first profile changes.

11. The surgical retractor device of claim 5, wherein the second profile defines a convex outer surface of the body.

12. The surgical retractor device of claim 11, wherein the second profile extends within the second plane between (i) the first side edge portion or the angled edge portion and (ii) the second transition edge portion or the second side edge portion of the peripheral edge of the body.

13. The surgical retractor device of claim 12, wherein the convex outer surface of the body defined by the second profile is continuously convex between (i) the first side edge portion or the angled edge portion and (ii) the second transition edge portion or the second side edge portion of the peripheral edge of the body.

14. The surgical retractor device of claim 1, wherein the body defines a height between (i) the first top edge portion or the second top edge portion and (ii) the bottom edge section of the peripheral edge of the body, the height between about 20 mm and about 60 mm.

15. The surgical retractor device of claim 1, wherein the body defines a width between (i) a point where the first side edge portion meets the angled edge portion and (ii) the second side edge portion of the peripheral edge of the body, the width between about 20 mm and about 60 mm.

16. The surgical retractor device of claim 1, wherein the body is made from a radiopaque material.

17. A surgical retractor device comprising:
a body defining:
a first profile within a first plane;
a second profile within a second plane perpendicular to the first plane;
a top edge section;
a side edge portion substantially perpendicular to the top edge section; and
a transition edge portion between the top edge section and the side edge portion,
wherein the first profile includes a curved portion, a flat portion, and a transition portion, wherein the curved portion of the first profile defines a convex outer surface of the body, wherein the transition portion of the first profile defines a concave outer surface of the body, and wherein the second profile defines a continuously convex outer surface of the body; and
a handle extending from at least one of the top edge section, the transition edge portion, or the side edge portion.

18. A method of performing a total ankle replacement surgery comprising:
making a first incision on a medial side of an ankle area of a patient;
inserting a retractor device into the patient via the first incision, the retractor device including a handle and a body having an outer convex surface;
adjusting a position of the retractor device towards a posterior side of a talus of the patient; and
inserting via a second incision on an anterior side of the ankle area of the patient a surgical instrument to perform a surgical procedure involving at least one of a calcaneus or the talus of the patient.

19. The method of claim 18, wherein the body includes a peripheral edge, wherein the peripheral edge includes:
a top edge section of the peripheral edge including a first top edge portion and a second top edge portion;
a first side edge portion substantially perpendicular to the first top edge portion;
a first transition edge portion between the first top edge portion and the first side edge portion, the first transition edge portion defining a first radius;
a bottom edge section;
an angled edge portion extending at an angle between the first side edge portion and the bottom edge section;
a second side edge portion opposite the first side edge portion and substantially perpendicular to the second top edge portion; and
a second transition edge portion between the second top edge portion and the second side edge portion, the second transition edge portion defining a second radius,
wherein the second radius of the second transition edge portion is larger than the first radius of the first transition edge portion; and
wherein the handle has a proximal end and a distal end, the distal end of the handle extending from at least one of the top edge section, the first transition edge portion, or the first side edge portion.

20. The method of claim 18, wherein the body defines:
a first profile within a first plane;
a second profile within a second plane perpendicular to the first plane;
a top edge section;
a side edge portion substantially perpendicular to the top edge section; and
a transition edge portion between the top edge section and the side edge portion,
wherein the first profile includes a curved portion, a flat portion, and a transition portion, wherein the curved portion of the first profile defines a convex outer surface of the body, wherein the transition portion of the first profile defines a concave outer surface of the body, and wherein the second profile defines a continuously convex outer surface of the body; and
wherein the handle extends from at least one of the top edge section, the transition edge portion, or the side edge portion.
